(19) United States
(12) Patent Application Publication
(10) Pub. No.: US 2009/0306525 A1
(43) Pub. Date: Dec. 10, 2009

(54) Apparatus and Method for Defibrillation Pulse Detection Using Electromagnetic Waves

(75) Inventors: Robert Pinter, Aachen (DE); Jens Muehlsteff, Aacjem (DE); Olaf Such, Aachen (DE); Jeroen Thijs, Aachen (DE)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., Eindhoven (NL)

(21) Appl. No.: 11/995,114

(22) PCT Filed: Jul. 5, 2006
(86) PCT No.: PCT/IB2006/052267

§ 371 Date: Jan. 9, 2008

(30) Foreign Application Priority Data
Jul. 15, 2005 (EP) 05106544.9
Aug. 26, 2005 (EP) 05107857.4

(51) Int. Cl. A61B 5/02

(52) U.S. Cl. 600/500

(57) ABSTRACT

A pulse detector that uses electromagnetic waves for detecting a patient pulse in conjunction with the administration of defibrillation and/or CPR. Electromagnetic waves are applied to a patient blood vessel and the reflected electromagnetic waves are analyzed for a Doppler shift, which is indicative of a pulse. In some applications the pulse detector can be used as a stand-alone device in conjunction with the administration of CPR. In other applications, the pulse detector is included with a defibrillator and provides pulsatile information that is analyzed in addition to ECG information in determining resuscitation therapy, or following defibrillation to ascertain its success.

In emergencies and during operative procedures, the assessment of the state of blood flow of the patient is essential for both diagnosis of the problem and determining the appropriate therapy for the problem. The presence of a cardiac pulse in a patient is typically detected by palpating the patient's neck and sensing palpable pressure changes due to the change in the patient's carotid artery volume. When the heart's ventricles contract during a heartbeat, a pressure wave is sent throughout the patient's peripheral circulation system. A carotid pulse waveform rises with the ventricular ejection of blood at systole and peaks when the pressure wave from the heart reaches a maximum. The carotid pulse falls off again as the pressure subsides toward the end of the pulse.

The absence of a detectable cardiac pulse in a patient is a strong indicator of cardiac arrest. Cardiac arrest is a life-threatening medical condition in which the patient's heart fails to provide blood flow to support life. During cardiac arrest, the electrical activity of the heart may be disorganized (ventricular fibrillation, VF), too rapid (ventricular tachycardia, VT), absent (asystole), or organized at a normal or slow heart rate without producing blood flow (pulseless electrical activity, PEA).

The form of therapy to be provided to a patient without a detectable pulse depends, in part, on an assessment of the patient's cardiac condition. For example, a caregiver may apply a defibrillation shock to a patient experiencing VF or VT to stop the unsynchronized or rapid electrical activity and allow a perfusing rhythm to return. External defibrillation, in particular, is provided by applying a strong electric shock to the patient's heart through electrodes placed on the patient's chest. If the patient lacks a detectable pulse and is experiencing asystole or PEA, defibrillation cannot be applied and the caregiver may perform cardiopulmonary resuscitation (CPR), which causes some blood to be forced through the cardiovascular system of the patient.

Before providing therapy such as defibrillation or CPR to a patient, a caregiver must first confirm that the patient is in cardiac arrest. In general, external defibrillation is suitable only for patients that are unconscious, apneic, pulseless, and in VF or VT. Medical guidelines indicate that the presence or absence of a cardiac pulse in a patient should be determined within 10 seconds. For example, the American Heart Association protocol for CPR requires a healthcare professional to assess the patient's pulse within five to ten seconds. Lack of a pulse is an indication for the commencement of external chest compressions. Assessing the pulse, while seemingly simple on a conscious adult, is the most often failed component of a basic life support assessment sequence, which may be attributed to a variety of reasons, such as lack of experience, poor landmarks, or error in either finding or not finding a pulse. Failure to accurately detect the presence or absence of the pulse can lead to adverse treatment of the patient either when providing or not providing CPR or defibrillation therapy to the patient.

Electrocardiogram (ECG) signals are normally used to determine whether or not a defibrillating shock should be applied. However, certain rhythms that a rescuer is likely to encounter cannot be determined solely by the ECG signal, e.g., pulseless electrical activity. Diagnoses of these rhythms require supporting evidence of a lack of perfusion despite the myocardial electrical activity as indicated by the ECG signal. Thus, in order for a rescuer to quickly determine whether or not to provide therapy to a patient, it is recommended that the patient's pulse and the ECG signals be analyzed in order to correctly determine the appropriate resuscitation therapy.

This necessity is particularly dire in situations or systems in which the rescuer is an untrained and/or inexperienced person, as is the case with rescuers for which the system described in U.S. Pat. No. 6,575,914 (Rock et al.) is designed. The '914 patent is assigned to the same assignee as the present invention and is hereby incorporated by reference in its entirety. The '914 patent discloses an automated external defibrillator (AED) (hereinafter both AEDs and semi-automated external defibrillators will be referred to jointly as AEDs) which can be used by first-responding caregivers with little or no medical training to determine whether or not to apply defibrillation to an unconscious patient.

The Rock AED has a defibrillator, a sensor pad for transmitting and receiving Doppler ultrasound signals, two sensor pads for obtaining an ECG signal, and a processor which receives and assesses the Doppler and ECG signals in order to determine whether defibrillation is appropriate for the patient (i.e., whether or not there is a pulse and the state of electrical cardiac activity) or whether another form of treatment such as CPR is appropriate. The Doppler pad is secured to a patient's skin above the carotid artery to sense the carotid pulse, which is a key indicator of the sufficiency of pulsatile blood flow. Specifically, the processor in the Rock AED analyzes the Doppler signals to determine whether there is a detectable pulse and analyzes the ECG signals to determine whether there is a “shockable rhythm.” Based on the results of these two separate analyses, the processor determines whether or not to advise defibrillation.

In addition to integrated Doppler ultrasound pulse detectors, clinicians currently use standalone Doppler ultrasound pulse detectors to detect the patient's pulse and to measure blood flow. Once the information is gathered by the Doppler system and processed, the rescuer then needs to gather the ECG signals and make a determination whether to defibrillate the patient.

Doppler ultrasound pulse detectors have a disadvantage in that an acoustic coupling medium, such as an ultrasound gel, is required to establish sufficient acoustic coupling to the patient. Thus, for ultrasound pulse detectors the ultrasound coupling gel needs to be available or packaged with the pulse detector. Packaging the ultrasound coupling gel with the pulse detector typically limits the detector to a one-time use, which is generally undesirable for cost reasons. Where ultrasound coupling gel is to be separately applied to the ultrasound pulse detector, application of the ultrasound gel takes time and adds yet another step is added to a process that in an emergency situation is already daunting to a lay rescuer.

In accordance with the principles of the present invention a pulse detector is provided for detecting a patient's pulse in conjunction with treatment for cardiac arrest such as defibrillation or the administration of CPR. The pulse detector includes a transmitter circuit operable to generate and emit electromagnetic waves and further includes a receiver circuit operable to detect and receive reflected electromagnetic waves. The receiver circuit is further operable to determine a frequency shift between the emitted electromagnetic waves and the reflected electromagnetic waves. An output circuit is coupled to the receiver circuit and operable to provide an indicator of the patient's pulse based on the frequency shift between the emitted electromagnetic waves and the reflected electromagnetic waves.

In an example of the present invention shown below a defibrillator system is provided having a defibrillator, electrodes, and a pulse detector. The defibrillator is operable to deliver defibrillation energy and the electrodes are coupled to the defibrillator to deliver the defibrillation energy through the electrodes. The electrodes are further configured to provide electrocardiogram (ECG) signals to the defibrillator. A pulse detector is coupled to the defibrillator and is configured to emit electromagnetic waves and generate a pulse signal provided to the defibrillator. The pulse signal from the pulse detector is based on a Doppler shift of reflected electromagnetic waves.

In another example of the present invention a method for detecting a patient pulse in conjunction with the administration of CPR is provided. The method includes applying electromagnetic waves to a patient blood vessel and determining the presence of a patient pulse based on a Doppler shift of the electromagnetic waves reflected from the patient blood vessel.

In another example of the present invention a method for delivering defibrillation energy to a patient is provided. The method includes monitoring an electrocardiogram (ECG) of the patient, applying electromagnetic waves to a patient blood vessel, and analyzing the ECG and electromagnetic waves reflected from the patient blood vessel to determine whether to deliver defibrillating energy to the patient. Prior to and/or subsequent to defibrillation the patients pulse is analyzed based on a Doppler shift of electromagnetic waves reflected from the carotid artery.

In the drawings:

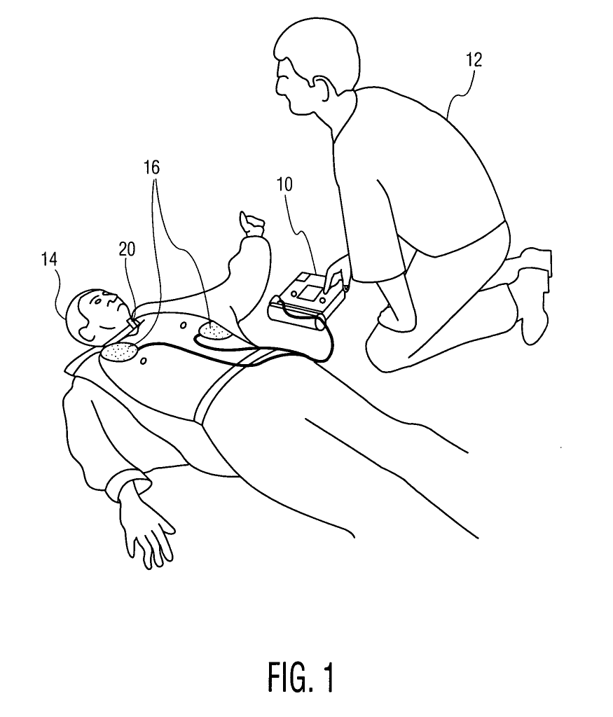

FIG. 1 is an illustration of a pulse detector according to an embodiment of the present invention and a defibrillator being applied to a patient suffering from cardiac arrest.

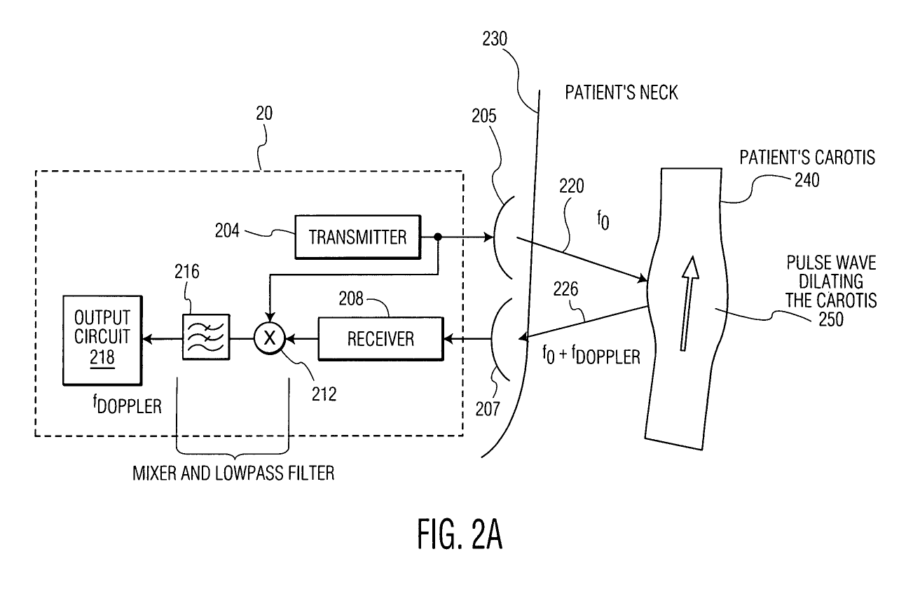

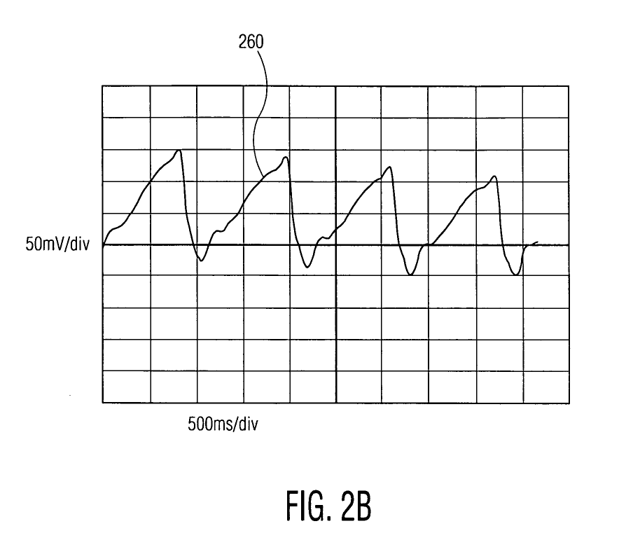

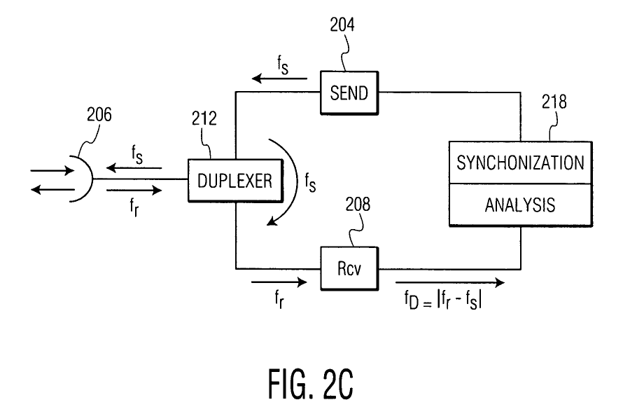

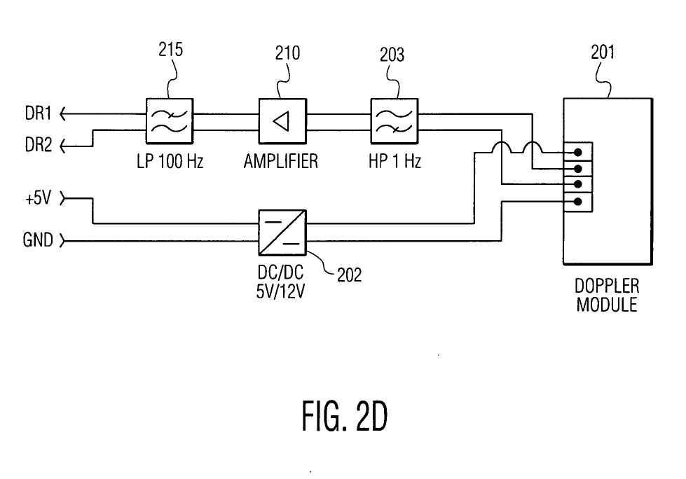

FIG. 2a is a simplified block diagram of the pulse detector of FIG. 1.

FIG. 2b illustrates a pulse indicator signal produced by the pulse detector of FIG. 2a.

FIG. 2c is a block diagram of another pulse detector constructed in accordance with the present invention.

FIG. 2d is a block diagram of another pulse detector constructed in accordance with the present invention.

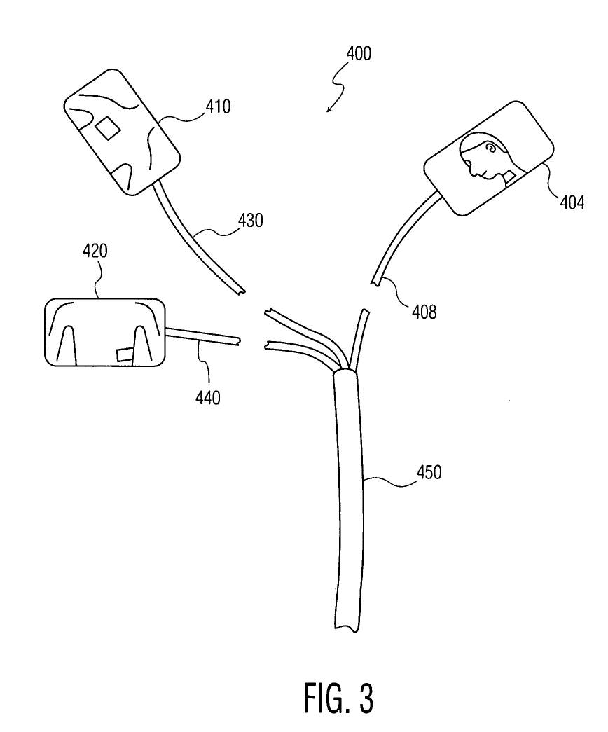

FIG. 3 is a diagram of cardiac resuscitation pad set according to an embodiment of the present invention.

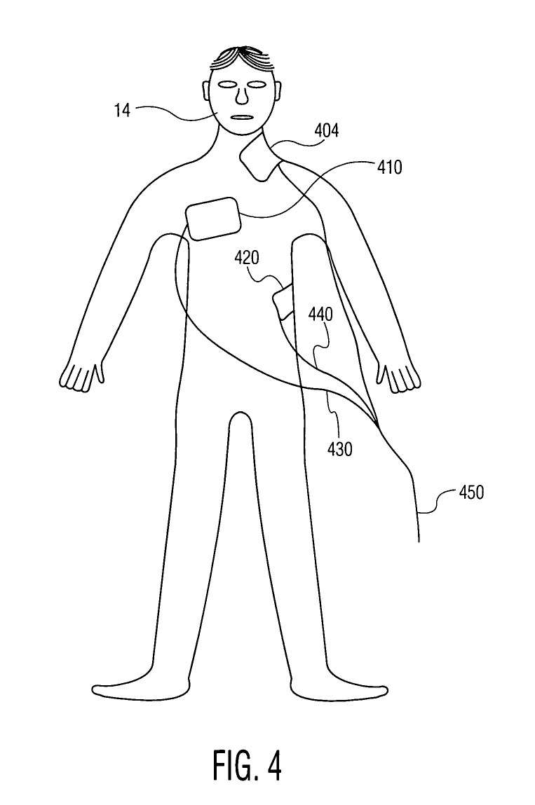

FIG. 4 is a diagram of placement of the cardiac resuscitation pad set on a patient.

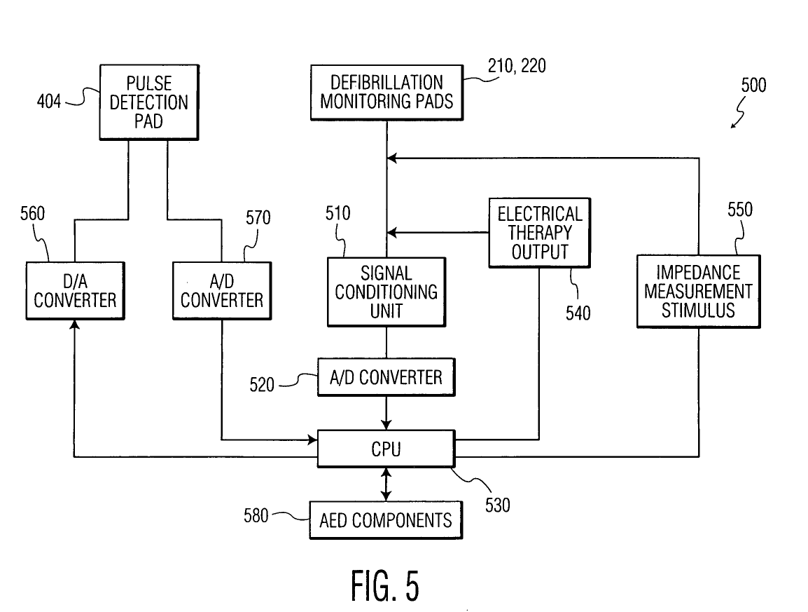

FIG. 5 is a simplified block diagram of a defibrillator for use with the cardiac resuscitation pad set.

Certain details are set forth below to provide a sufficient understanding of the invention. However, it will be clear to one skilled in the art that the invention may be practiced without these particular details. Moreover, the particular embodiments of the present invention described herein are provided by way of example and should not be used to limit the scope of the invention to these particular embodiments. In other instances, well-known circuits, control signals, timing protocols, and software operations have not been shown in detail in order to avoid unnecessarily obscuring the invention.

FIG. 1 is an illustration of a pulse detector 20 according to an embodiment of the present invention and an AED 10 applied to resuscitate a patient 14 suffering from cardiac arrest. A pair of electrodes 16 coupled to the AED 10 are applied across the chest of the patient 14 by a rescuer 12 in order to acquire an ECG signal from the patient's heart. The pulse detector 20 is positioned on the patient's neck proximate to the patient's carotid artery to sense the carotid pulse. As will be described in more detail below, the pulse detector 20 uses electromagnetic waves for detecting a patient's pulse. In contrast to some conventional pulse detectors, the pulse detector 20 can be applied to the patient 14 without a coupling medium, such as coupling gel. A neck ruff or Velcro strap or adhesive substrate to which the pulse detector 20 is attached can be used to position the pulse detector on the patient 14.

As previously discussed, the combination of patient pulsatility and ECG conditions can be used to determine the therapy that should be administered by the rescuer 12. For example, in sudden cardiac arrest, the patient 14 is stricken with a life threatening interruption to the normal heart rhythm, typically in the form of VF or VT that is not accompanied by a palpable pulse (i.e., shockable VT). The defibrillator 10 analyzes the ECG signal for signs of arrhythmia. If a treatable arrhythmia is detected, the defibrillator 10 signals the rescuer 12 that a shock is advised, and the rescuer 12 is prompted to press a shock button on the defibrillator 10 to deliver defibrillation pulse to resuscitate the patient 14. However, where a pulse is detected, or no pulse is detected and a shockable rhythm is not present, as determined by analysis of the pulse signal and the ECG signal by the AED 10, defibrillation should not be applied and the rescuer 12 should perform CPR on the patient 14 instead. The pulse detector 20 will monitor the pulsatile flow of blood to the head during the administration of CPR and remains in place to assess the advisability of defibrillation after the CPR period is over.

FIG. 2a illustrates the pulse detector 20 according to an embodiment of the present invention and the physiological interaction with the patient 14. The block diagram of the pulse detector 20 is simplified, illustrating components that will be described in more detail below. Those ordinarily skilled in the art, however, will appreciate that other well-known components are included in the pulse detector 20 as well.

The electromagnetic waves generated by the detector 20 penetrate the throat and are reflected at boundary layers between areas of different electrical conductivity. Inside the human body, blood vessels represent areas with an electrical conductivity that is significantly higher than the electrical conductivity in the region surrounding the blood vessels. As a result, electromagnetic waves propagating inside the human body will primarily be reflected by large blood vessels, such as the carotid artery, which is the artery that supplies the head with blood and is also located close to the skin surface.

As is known, blood vessels such as the carotid artery dilate in response to a pulse wave as blood is pumped to the head. The reflected electromagnetic waves can be used to detect a patient pulse because blood flow inside the blood vessels cause periodic dilation of the blood vessels. As a result, when a pulse is present the electromagnetic waves reflected by the blood vessel undergo a Doppler shift, which can be detected and used to determine whether the patient has a pulse. That is, movement (periodical dilation) of the carotid artery is indicative of a pulse, whereas a lack of movement is indicative of no pulse. Movement of the carotid artery is determined by the presence of a Doppler shift in the reflected electromagnetic wave.

The electromagnetic waves do not propagate along a single ray as FIG. 2a may suggest, but instead can be represented by a lobe pattern covering an area in front of the antenna. Due to the width of the lobe, the pulse detector can be generally positioned proximate to the carotid artery and still detect motion. By investigating the frequency of the reflected electromagnetic waves with respect to the frequency of the emitted wave it is possible to obtain information on the movement of the carotid artery wall towards the main lobe of the emitted electromagnetic waves. The information obtained from this measurement is correlated to the mechanical motion of the carotid artery wall.

With reference to FIG. 2a, the detector 20 includes a transmit circuit 204 that emits electromagnetic waves 220 into the patient's neck 230 through an antenna 205. A receiver circuit 208 receives electromagnetic waves 226 reflected by the patient's carotis 240 and surrounding area through an antenna 207. In an example illustrated below the transmitting antenna 205 and the receiving antenna 207 are realized as a single antenna serving both transmitting and receiving functions. A mixing circuit 212 and lowpass filter 216 are used to demodulate and detect the reflected electromagnetic wave 226 and provide an output signal exhibiting the Doppler frequency f_{Doppler}. An output circuit 218 receives the output signal and generates a pulse indicator that is used to inform the rescuer 12 whether a pulse is detected. A typical pulse indicator signal 260 is shown in FIG. 2b. A power supply (not shown) provides power to the circuitry of the detector 20 for operation. In some embodiments of the present invention, the transmitter circuit 204, receiver circuit 208, antennas 205, 207, mixer 212, and lowpass filter 216 are integrated in a microwave sensor package, such as those known in the art for motion detection, an example of which is discussed below. Such motion sensors utilize electromagnetic waves having frequencies in the gigahertz range, for example, ranging from 1.2 GHz, 2.45 GHz to 12 GHz and 22 GHz.

The main lobe of the emitted electromagnetic waves 220 is directed towards the patient's carotis 240. The frequency of the emitted electromagnetic waves 220 is nominally f₀. Upon reflection from the carotis 240, a frequency shift is introduced if a pulse wave 250 causes the carotis 240 to dilate or contract. As a result, the reflected electromagnetic waves 226 have a frequency (f₀+f_{Doppler}) that is shifted with respect to f₀ of the emitted electromagnetic waves 220. The frequency shift f_{Doppler} is related to the velocity of the carotis 240 by the following equation.

$${f}_{Doppler} = \pm {f}_{0} \cdot \frac{2 \cdot v}{c}$$

with c equal to the velocity of light and v being the velocity of the carotis 240 approaching or receding relative to the transmitter 204/receiver 208. A dilating carotis 240 expanding toward the transmitter circuit 204/receiver circuit 208 results in a positive frequency shift (i.e., +f_{Doppler}) and a contracting carotis 240 (as the pulse wave 250 propagates past) receding from the transmitter circuit 204/receiver circuit 208 results in a negative frequency shift (i.e., −f_{Doppler}). Where no blood is being pumped through the patient's carotis 240 (i.e., no pulse), the carotis 240 is relatively motionless with respect to the transmitter 204/receiver 208 and little or no shift in frequency is introduced in the reflected electromagnetic waves 220.

Based on the output signal from the lowpass filter 216, the output circuit 218 generates a pulse indicator signal that can be interpreted by the rescuer 12 as indicating the presence or absence of a pulse. For example, where the output circuit 218 includes an audible speaker, audible output information such as a simulated heart beat can be generated by the output circuit 218 that is indicative of a pulse. Visual output information can be additionally or alternatively provided where the output circuit 218 includes a display. The output circuit 218 can display a pulsing illumination corresponding to the patient's pulse, or a numerical value corresponding to the patient's pulse rate can be displayed.

FIG. 2c is a block diagram of another pulse detector constructed in accordance with the present invention. The transmitter electronics 204 instruct a duplexer 212 to send electromagnetic signals of frequency fₛ which are emitted by an antenna 206. Reflected electromagnetic signals Doppler shifted to frequency fᵣ are received by the antenna 206, passed back to the duplexer 212 and coupled to receiver processing electronics 208 which receives the signal and calculates f_{D} the shift in frequency between the emitted and received electromagnetic signals by a mixing process of |fᵣ−fₛ|, as is known in the art. Although a single antenna 206 is shown here, separate antennas may be used for sending and receiving the electromagnetic signals. The shift in frequency f_{D} is communicated to the output circuit 218 which performs synchronization and analysis of the received Doppler information.

FIG. 2d is a block diagram of another pulse detector constructed in accordance with the present invention. This example utilizes a commercially available Microwave Motion Sensor KMY 24 module made by Micro Systems Engineering GmbH of Berg, Germany. This device contains a 2.45 GHz oscillator and receiver in the same housing and operates in a continuous wave mode. The dimensions of the beam are, inter alia, dependent on the dimensions of the antenna and in this example the module contains an optimized patch antenna with minimized dimensions and a width of 3.5 cm, producing a beam with a near field radius of 2 cm. This provides a workable compromise between too small an antenna, which would produce a wide beam easily contaminatable by reflections from a variety of tissue structures in the throat, and too large an antenna which would produce a narrow beam which may fail to intercept the carotid artery.

This module is utilized in the following way. FIG. 2d shows a block diagram of the apparatus. The Doppler module 201 is powered by a voltage supply 202. The output of the Doppler module 201 is processed through a high pass filter 203, a preamplifier 210 and a low pass filter 215. In an experimental embodiment the high pass filter 203 employed a capacitance of 100 nF and a resistor of 1 MΩ, as this enabled a faster decay of the signal while removing the DC part of the signal from the Doppler module. The time constant τ of 0.1 s produces a cut-off frequency of 1.59 Hz. Although the signal being detected is reflected from the carotid artery which pulses at a frequency of the order of 1 Hz in a conscious individual, the attenuation of this first order high pass filter is low enough not unduly attenuate the signal. The gain of the preamplifier 210 can be set in a range of 1 to 1000 but it was found that a particularly advantageous gain was 500. To enable sampling, an 8^{th} order low pass filter 215 was realized with a cutoff frequency of 100 Hz using operational amplifiers.

FIG. 2d also shows two output signals, DR1 and DR2, from the Doppler module. As is known in the art, some commercially available transducers contain two mixer diodes to provide additional information about the direction of movement of the reflecting object, e.g., toward or away from the module. However, two signals are not necessary in a particular implementation of the present invention. If such a module is used to construct the inventive apparatus the reflected signal from either mixer diode can be used for the calculation of the rate of change of the received signal characteristic of pulsatility.

FIG. 3 illustrates a cardiac resuscitation pad set 400 according to an embodiment of the present invention. The pad set 400 is connected to a defibrillator 500 (FIG. 5) to form a cardiac resuscitation system. The pad set 400 includes a pulse detection pad 404 and defibrillation-monitoring pads 410, 420. The pulse detection pad 404 includes a pulse detector according to an embodiment of the present invention. In one embodiment, the pulse detection pad 404 includes the pulse detector 20 previously described with reference to FIGS. 1 and 2.

Conductors 408, 430, 440 from the pulse detection pad 404 and the defibrillation-monitoring pads 410, 420 are connected to the defibrillator 500 by cable 450. To assist a rescuer 12 in properly placing the pulse detection pad 404 and the defibrillation-monitoring pads 410, 420, a pictorial instruction can be included on each pad. For example, as shown in FIG. 3, each of the defibrillation-monitoring pads 410, 420 includes a picture of a human torso indicating the location the defibrillation-monitoring pads 410, 420 should be placed on the torso. Similarly, the pulse detection pad 404 includes a diagram of the patient's neck and the location at which the pulse detection pad 404 should be applied. As previously discussed, the pulse detection pad 404 should preferably be applied proximate to the patient's carotis.

FIG. 4 illustrates placement of the cardiac resuscitation pad set 400 including the defibrillation-monitoring pads 410, 420 and the pulse detection pad 404 on a patient 14. The pulse detection pad 404 is applied to the patient's neck to sense the carotid pulse and the defibrillation-monitoring pads 410, 420 are applied to the patient's torso as shown. In the illustrated example, the pulse detection pad 404 is adhered to the patient's neck and the defibrillation-monitoring pads 410, 420 are adhered to the body using conventional medical adhesives. A conductive gel is included with the defibrillation-monitoring pads 410, 420 to electrically couple the patient's skin to the pads 410, 420.

The cardiac resuscitation pads 400 are utilized with a defibrillator that analyzes both the patient's ECG and pulse in determining the proper therapy to be applied by the rescuer 12. The defibrillation-monitoring pads 410, 420 are used to couple ECG signals to the defibrillator, which are analyzed to determine whether the patient's heart is undergoing a shockable rhythm. Defibrillation therapy is also delivered using the defibrillation-monitoring has pads 410, 420, if necessary. The pulse detection pad 404 is used to provide pulse detection signals to the defibrillator 500 for determining whether the patient 14 has a pulse, which is indicative of blood flow.

FIG. 5 illustrates a defibrillator 500 that analyzes both pulse detection and ECG signals, and determines whether a pulse is detected and whether a shockable rhythm is present. The defibrillation-monitoring pads 210, 220 detect and provide the ECG signals from the patient 14 to the defibrillator 500. A signal conditioning unit 510 conditions the ECG signals by filtering the ECG signals. An analog-to-digital (A/D) converter 520 converts the conditioned ECG signals to digital signals and provides the digital signals to a central processing unit (CPU) 530 for analysis. The CPU 530 includes permanent or removable storage, such as magnetic and optical discs, RAM, ROM, and the like, on which the process and data structures can be stored and distributed. The CPU 530 may further perform an impedance measurement stimulus 550 by sending very high frequency signals across the defibrillation-monitoring pads 210, 220. The impedance measurement stimulus is recorded in the signal conditioning unit 510 thereby providing a rescuer with the ability to determine if the defibrillation-monitoring pads 210, 220 are making good contact with the patient.

The CPU 530 also outputs digital signals and transmits the digital signals to the pulse detection pad 404 via a digital-to-analog (D/A) converter 560. Analog signals from the D/A converter 560 trigger a transmitter circuit of the pulse detection pad 404 to emit electromagnetic waves into the patient. In some implementations the D/A converter may not be needed and a digital signal used to trigger the transmitter circuit of the pulse detector The reflected electromagnetic waves are received by a receiver circuit in the pulse detection pad 404 and an A/D converter 570 converts the pulse indicator signal produced by the pulse detector output circuit to a digital signal. The digital pulse indicator signal is provided to the CPU 530 for further processing to determine whether a pulse is present. AED components 580 includes necessary hardware for a complete defibrillation system, that is, for example, memory, program storage, result storage, user interface elements, such as buttons, audio system and speaker, and a power supply.

Based on the ECG and pulse information obtained through the defibrillation-monitoring pads 210, 220 and the pulse detection pad 404, respectively, the CPU 530 determines the appropriate therapy for the patient 14. For example, if the CPU 530 determines that a defibrillation therapy should be applied to the patient 14, then the CPU 530 charges the energy storage capacitor (not shown) to apply an electrical therapy output pulse 540 to the patient 14 via the defibrillation-monitoring pads 210, 220. Alternatively, if the information based on the pulse and ECG of the patient 14 indicates administering CPR, audible commands can be given over an audible speaker included in the AED components 580 for instructing a rescuer to administer CPR to the patient 14.

From the foregoing it will be appreciated that, although specific embodiments of the invention have been described here in for purposes of illustration, various modifications may be made without deviating from the spirit and scope of the invention. Accordingly, the invention is not limited except as by the appended claims.

1. A pulse detector for detecting a patient's pulse during administration of CPR, comprising:
a transmitter circuit operable to generate and emit electromagnetic waves;
a receiver circuit operable to detect and receive reflected electromagnetic waves and further operable to determine a frequency shift between the emitted electromagnetic waves and the reflected electromagnetic waves; and
an output circuit coupled to the receiver circuit and operable to provide an indicator of the patient's pulse during CPR based on the frequency shift between the emitted electromagnetic waves and the reflected electromagnetic waves.

2. The pulse detector of claim 1, further including an attachment device configured to position the pulse detector in proximity to the carotid artery of the patient.

3. The pulse detector of claim 1 wherein the pulse detector is included in an adhesive pad configured to be adhered to the patient during the administration of CPR.

4. The pulse detector of claim 1 wherein the transmitter circuit comprises a transmitter circuit operable to generate and emit electromagnetic waves having a frequency of at least 1.0 gigahertz.

5. The pulse detector of claim 1 wherein the receiver circuit comprises a receiver, a mixer circuit and a lowpass filter circuit.

6. The pulse detector of claim 1 wherein the output circuit comprises an output circuit configured to provide an audible indicator of the patient's pulse during CPR based on the frequency shift.

7. The pulse detector of claim 1 wherein the output circuit comprises an output circuit configured to provide a visual indicator of the patient's pulse during CPR based on the frequency shift.

8. A defibrillator system, comprising:
a defibrillator operable to deliver defibrillation energy;
electrodes coupled to the defibrillator and configured to deliver the defibrillation energy through the electrodes, the electrodes further configured to provide electrocardiogram (ECG) signals to the defibrillator; and
a pulse detector coupled to the defibrillator and configured to emit and receive electromagnetic waves and generate a pulse signal provided to the defibrillator, the pulse signal based on a Doppler shift of reflected electromagnetic waves.

9. The defibrillator system of claim 8 wherein the electrodes and the pulse detector are coupled to the defibrillator through a common cable.

10. The defibrillator system of claim 8 wherein the pulse detector comprises:
a transmitter circuit operable to generate and emit electromagnetic waves;
a receiver circuit operable to detect and receive reflected electromagnetic waves and further operable to determine a frequency shift between the emitted electromagnetic waves and the reflected electromagnetic waves.

11. The defibrillator system of claim 10 wherein the pulse detector further comprises an output circuit coupled to the receiver circuit and operable to provide an indicator of the patient's pulse based on the frequency shift between the emitted electromagnetic waves and the reflected electromagnetic waves.

12. The defibrillator system of claim 8 wherein the defibrillator comprises a defibrillator configured to monitoring the ECG of a patient and analyze the ECG and pulse signal to determine whether to deliver defibrillating energy to the patient.

13. The defibrillator of claim 8 wherein the defibrillator comprises an automatic external defibrillator.

14. A method for monitoring a patient pulse during administration of CPR, comprising:
applying an electromagnetic transducer in the vicinity of the carotid artery;
applying electromagnetic waves to the carotid artery;
performing CPR; and
determining the presence of a patient pulse based on a Doppler shift of the electromagnetic waves reflected from the carotid artery.

15. The method of claim 14 wherein applying electromagnetic waves to the carotid artery comprises applying electromagnetic waves having a frequency of at least 1.0 gigahertz.

16. The method of claim 14, further comprising generating a visual indicator of arterial pulsatility in response to the determination.

17. The method of claim 14, further comprising generating an audible indicator of arterial pulsatility in response to the determination.

18. A method for delivering defibrillation energy to a patient, comprising:
monitoring an electrocardiogram (ECG) of the patient;
applying electromagnetic waves to a carotid artery; and
analyzing the ECG and electromagnetic waves reflected from the carotid artery to determine whether to deliver defibrillating energy to the patient.

19. The method of claim 18 wherein analyzing the electromagnetic waves reflected from the carotid artery comprises determining a Doppler shift in the reflected electromagnetic waves.

20. The method of claim 19 wherein analyzing the electromagnetic waves reflected from the carotid artery comprises determining from the Doppler shift in the reflected electromagnetic waves the presence of a patient pulse.

21. The method of claim 18 wherein analyzing the ECG comprises determining from the ECG the presence of at least one of ventricular fibrillation and ventricular tachycardia.

22. The method of claim 18 wherein applying electromagnetic waves to the carotid artery comprises applying electromagnetic waves having a frequency of at least 1.0 gigahertz to the patient blood vessel.

23. A method for delivering defibrillation energy to a patient, comprising:
monitoring an electrocardiogram (ECG) of the patient;
analyzing the ECG to determine whether to deliver defibrillating energy to the patient; and
following delivery of defibrillating energy, analyzing the Doppler shift of electromagnetic waves received from a blood vessel of the patient to determine whether defibrillation was successful.

24. The method of claim 23, wherein analyzing further comprises analyzing the ECG and the pulsatility of the Doppler shift of electromagnetic waves received from a blood vessel of the patient to determine whether to deliver defibrillating energy to the patient.
